(19) United States
(12) Patent Application Publication
(10) Pub. No.: US 2008/0077011 A1
(43) Pub. Date: Mar. 27, 2008

(54) ULTRASONIC APPARATUS

(76) Inventors: TAKASHI AZUMA, Kawasaki (JP); Hideki Yoshikawa, Kokubunji (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/838,263

(22) Filed: Aug. 14, 2007

(30) Foreign Application Priority Data
Sep. 27, 2006 (JP) 2006-262603

(51) Int. Cl. A61B 8/08

(52) U.S. Cl. 600/443

(57) ABSTRACT

An edge between a tumor and a normal tissue is detected even when acoustic impedance and elasticity of those are not changed. An edge position of tissue is estimated by setting a plurality of estimation regions of an inspection object, detecting direction of motion of the inspection object within each estimation region, and computing the point of inflexion in the direction of motion. Moreover, these edge positions are overlapped on the cross-sectional images and thereby an operator can easily detect the edge lines.

### CLAIM OF PRIORITY

The present application claims priority from Japanese application JP 2006-262603 filed on Sep. 27, 2006, the content of which is hereby incorporated by reference into this application.

### FIELD OF THE INVENTION

The present invention relates to an ultrasonic apparatus for displaying ultrasonic cross-sectional images.

### BACKGROUND OF THE INVENTION

An ordinary ultrasonic apparatus of the prior art includes an ultrasonic transducing unit for transmitting and receiving ultrasonic wave to an analyte, a cross-sectional scanning unit for repeatedly obtaining cross-sectional data in the predetermined period within the analyte including moving tissue using a reflection echo signal from such ultrasonic transducing unit, and an image displaying unit for displaying time series cross-sectional images obtained with such cross-sectional scanning unit. The information having converted a degree of non-continuity into luminance at the interface where acoustic impedance along the propagating direction of sound changes among a structure of the moving tissue within the analyte has been displayed as a B mode image.

Meanwhile, a method for obtaining an elastic image on the basis of data of elasticity by applying an external force from the surface of the analyte to assume a curve of attenuation of such external force within the living body and then measuring elasticity by obtaining pressure and displacement at each point from the assumed attenuation curve has been proposed in Ultrasonic Imag., vol. 13, pp. 111-134, 1991 by J. Ophir et al.

According to such elastic image, a degree of hard and soft tissues in the living body can be measured and displayed. Particularly, in a tissue which is different in the property from a peripheral tissue such as tumor, sound velocity in the vertical wave results, in some cases, in a large difference in the sound velocity in the lateral wave even if difference from the peripheral tissue is rather small. In this case, change in acoustic impedance does not appear in an image disabling discrimination on the B mode image but elasticity changes because sound velocity in the lateral wave changes and thereby such change in the acoustic impedance can be discriminated in some cases on the elastic image.

### SUMMARY OF THE INVENTION

However, tumors are formed in various properties and shapes and not only acoustic impedance but also elasticity doe not different to a large extent from the peripheral tissue depending on the tumors generated. In this case, however, an edge of image from the peripheral tissue could not be displayed as an image in some ultrasonic images even if using any of the B mode image and elastic image in the prior art. For example, in the case where the center of tumor is sphacelated, the sphacelated part is lowered in the luminance in the B mode image and existence itself of tumor cannot be detected because the sphacelated part becomes soft even in the elastic image. However, since a part requiring to a maximum extent the diagnosis, not yet being sphacelated at the edge of tumor, and being active as the carcinoma cell does show clear edge because a difference from the peripheral normal tissue surrounding the tumor is rather small in both acoustic impedance and elasticity. If the edge becomes unclear, it becomes difficult to determine the diagnostic area for low invasive diagnosis such as radioactive diagnosis, RF diagnosis, and ultrasonic diagnosis and moreover if change in the size of tumor cannot be assumed accurately, selection of medication in the diagnosis with an anti-carcinoma medication becomes difficult. From the viewpoints explained above, it is required to propose a new ultrasonic imaging method to detect acoustic impedance and elasticity even when these are not changed.

It is therefore an object of the present invention to provide an ultrasonic apparatus for solving the problems explained above.

The present invention attains the object explained above by comprising an ultrasonic cross-sectional image acquirer for acquiring on the time series basis plural frames of the ultrasonic cross-sectional images of the inspection object, a memory for storing the ultrasonic cross-sectional images of plural frames obtained, a motion detector for extracting information about movement of each tissue within the ultrasonic cross-sectional image of a first frame through comparison of the ultrasonic cross-sectional image of the first frame read from the memory with the ultrasonic cross-sectional image of a second frame, and edge detector for detecting the edge within the ultrasonic cross-sectional image on the basis of the information about the motion detected with the motion detector, and a display for displaying the edge detected with the edge detector overlapping on the ultrasonic cross-sectional image obtained with the ultrasonic cross-sectional image acquirer.

According to one aspect of the present invention, the motion detector sets respectively plural measuring regions on the ultrasonic cross-sectional image of the first frame and the ultrasonic cross-sectional image of the second frame read from the memory, detects, with pattern matching, the measuring region of the first frame and the measuring region of the second frame, and extracts direction and amplitude of motion of each tissue from relative position of the measuring region of the first frame and the measuring region of the second frame matched with the measuring region of the first frame. The edge detector obtains an edge by executing the threshold value process to the image formed on the scalar quantity extracted from the information about motion of each tissue in the ultrasonic cross-sectional image.

According to another aspect of the present invention, the motion detector sets respectively plural measuring regions on the ultrasonic cross-sectional image of the first frame and the ultrasonic cross-sectional image of the second frame read from the memory, and detects a correlation value of the measuring region of the first frame and the measuring region of the second frame matched with the measuring region of the first frame through the pattern matching by expanding the size of measuring region of the second frame in the predetermined direction in view of obtaining the measuring region when the correlated value shows the peak value. The edge detector detects the edge by defining a crossing point of the measuring region when the correlation value shows the peak value and the predetermined direction as the point of inflexion and then connecting plural points of inflexion.

According to the present invention, the edge of the tumor and normal tissue can be detected even if acoustic impedance and elasticity are not changed. Moreover, the area and volume of the region surrounded with the edges can be calculated.

### BRIEF DESCRIPTION OF THE DRAWING

FIG. 1 is a block diagram showing an apparatus structure for embodying the present invention;

FIG. 2 is a processing flow diagram for embodying the present invention;

FIGS. 3A and 3B are explanatory diagrams of a motion vector estimating method;

FIGS. 4A and 4B are explanatory diagrams of the motion vector estimating method for embodying the present invention;

FIGS. 5A, 5B, 5C, 5D, 5E, and 5F are explanatory diagrams for a method of setting motion estimation regions for embodying a first embodiment of the present invention;

FIG. 6 includes diagrams for explaining edge detecting results;

FIGS. 7A, 7B, 7C, 7D, and 7E are diagrams for explaining an edge estimating method in the first embodiment;

FIGS. 8A, 8B, and 8C are diagrams for explaining the edge estimating method in the first embodiment;

FIG. 9 is a block diagram showing an apparatus structure for embodying the present invention;

FIG. 10 is a processing flow diagram for embodying a second embodiment;

FIGS. 11A and 11B are diagrams for explaining a motion vector estimating method for embodying the second embodiment;

FIG. 12 is a diagram for explaining the edge point estimating method in the second embodiment;

FIGS. 13A and 13B are diagrams for explaining a method of setting motion estimation region in the second embodiment;

FIG. 14 is a diagram for explaining the method of setting motion estimation region in the second embodiment;

FIGS. 15A, 15B, 15C, and 15D are diagrams for explaining relationship between sharpness of edge and property and shape of tissue in a third embodiment;

FIG. 16 includes diagrams for explaining edge extraction by means of summing of frames;

FIG. 17 includes diagrams for explaining discontinuity and blurring of edge due to simple summing;

FIG. 18 includes diagrams for explaining edge extraction in a fourth embodiment;

FIG. 19 is a flowchart showing procedures for summing of motion compensating frames; and

FIGS. 20A and 20B are diagrams showing relationship between the motion measuring regions and searching regions.

### DESCRIPTION OF THE PREFERRED EMBODIMENTS

The preferred embodiments of the present invention will be explained below with reference to the accompanying drawings.

### First Embodiment

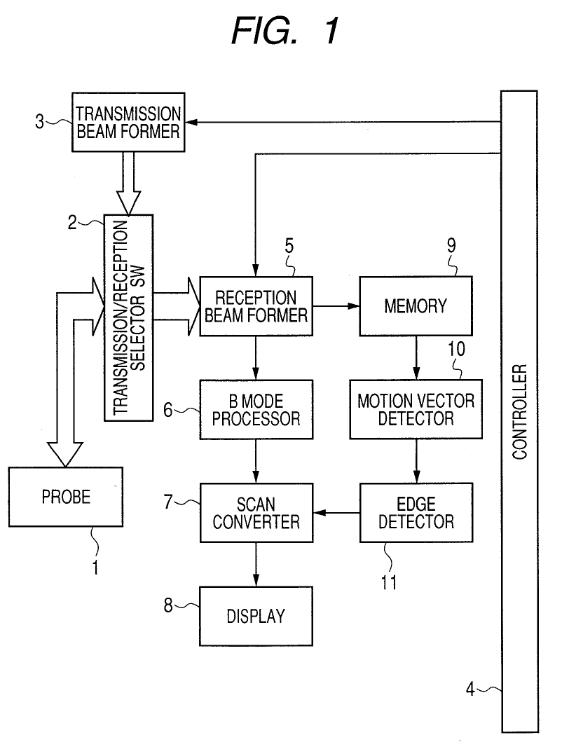

FIG. 1 is a block diagram showing an example of structure of an ultrasonic apparatus of the present invention. Flow of signal processes for display of image on the ultrasonic apparatus will be explained with reference to FIG. 1. A transmission beam former 3 sends a transmission electric pulse to an ultrasonic probe 1 preset on the front surface of an analyte via a transmission/reception selector 2 under the control of a controller 4. In this timing, the transmission beam former controls a delay time among channels of the probe 1 to the adequate state to permit the ultrasonic beam travel on the predetermined scanning line. The electrical signal from this transmission beam former 3 is converted into the ultrasonic signal with the ultrasonic probe 1 and thereby an ultrasonic pulse is transmitted into the analyte. The ultrasonic pulse scattered within the analyte is partly received again with the ultrasonic probe 1 as an echo signal and such received ultrasonic signal is converted into an electric signal. The electric signal converted from the ultrasonic signal is then supplied to a reception beam former 5 via the transmission and reception selector 2. Here, the electrical signal is converted to the data on the scanning line, where the echo signal from the desired depth on the predetermined scanning line is selectively enhanced, and is then stored in a memory 9. The data once accumulated in the memory is then subjected to correlational arithmetic operation between the frames in a motion vector detector 10 in order to compute motion vector. Edge among internal organs and that among tumor and normal tissue determined from motion within a notable image on the basis of the computed motion vector are detected in an edge detector 11. Meanwhile, the data from the reception beam former 5 is converted into an envelope signal from the RF signal in a B mode processor 6, then converted into an Log-compressed B mode image, and is then transmitted to a scan converter 7. On the scan converter 7, the visualized edge information and the B mode image are overlapped with each other for scan conversion. The data after the scan conversion is sent to a display 8 and is then displayed as an ultrasonic cross-sectional image on the display 8.

Processes in the motion vector detector 10 and edge detector 11 and processes other than that for superimposing the results of above processes to the B mode image on the scan converter 7 are executed with the ordinary ultrasonic apparatus. Accordingly, detail explanation of such processes is omitted here. Only detection of motion vector and detection of edge will be explained below.

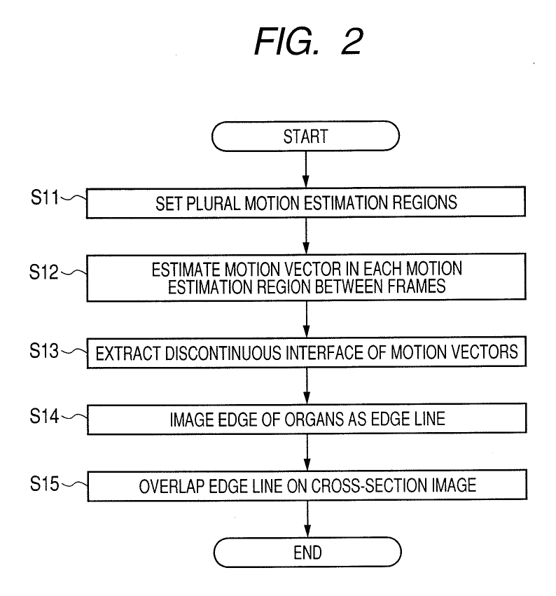

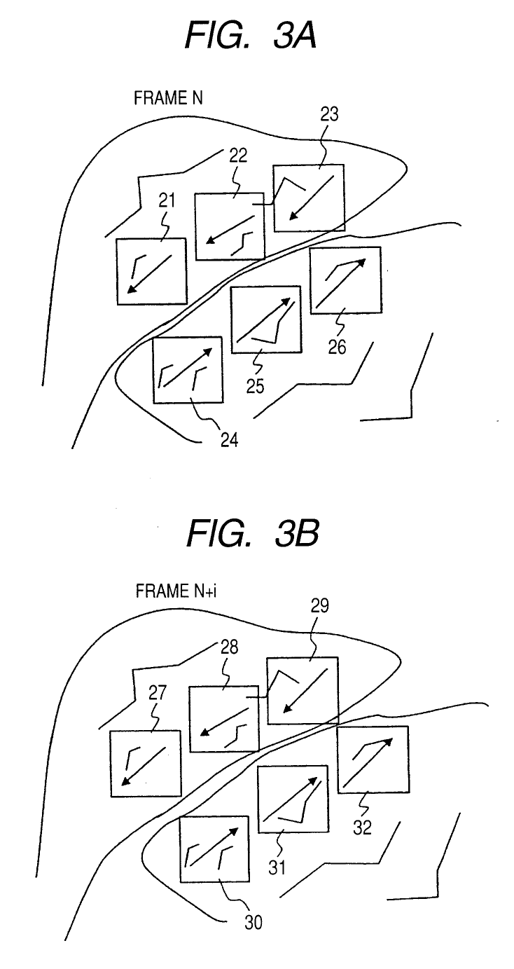

Flow of processes in this embodiment will be explained with reference to FIG. 2. First, a frame image is divided into plural motion estimation regions (S11) in order to obtain a motion vector. The reason of division into plural motion estimation regions is that if mutual correlation is obtained for a large region before the division, it becomes impossible to accurately estimate the motion when correlation becomes bad due to deformation. Therefore, it is preferable that the motion estimation region is as small as providing identical motion within the measuring regions. However, if such region is too small, characteristics of images are lost and correlation with every place can be obtained. In general, it is preferable to provide the motion estimation region as small as possible within the range larger than a speckle size (ultrasonic beam size). In the case of obtaining correlation between a frame N and a frame N+i, a motion estimation regions are respectively set on the image of frame N and on the image of frame N+i. FIG. 3A is a diagram showing the motion estimation regions 21 to 26 preset on an ultrasonic cross-sectional image of the frame N, while FIG. 3B is a diagram showing the motion estimation regions 27 to 32 preset on an ultrasonic cross-sectional image of the frame N+i. Here, i is set in accordance with velocity of motion of an object and when motion velocity is high, i is reduced and when search is carried out for the region where motion velocity is rather slow, a large integer is selected as a value of i.

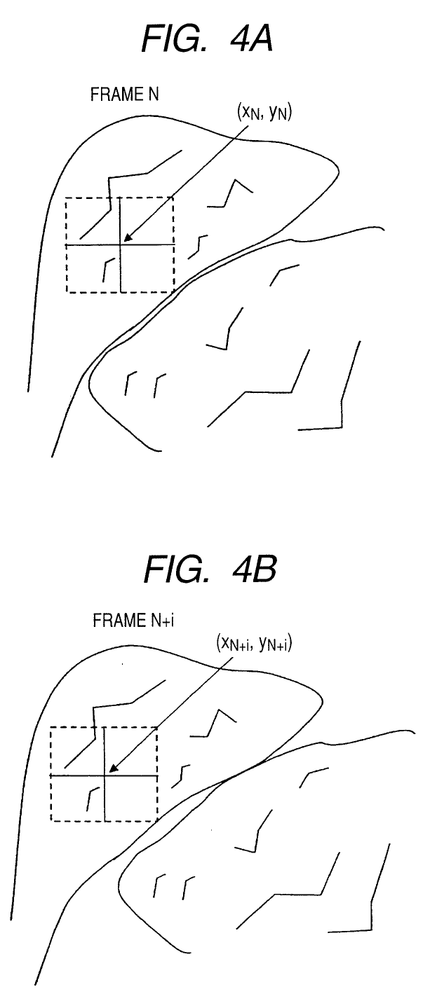

Next, a motion vector is detected with mutual correlation between the motion estimation regions 21 to 26 set on the ultrasonic cross-sectional image of the frame N and the motion estimation regions 27 to 32 set on the ultrasonic cross-sectional image of the frame N+i (or with the other method used widely for pattern matching such as least square method) (FIG. 2, S12). The motion vector is defined as follows. As is shown in FIGS. 4A and 4B, when the central point of the motion vector measuring region set in the frame N is defined as (x_{N}, y_{N}), while the central point of the region best matched with the motion estimation region of the frame N in the frame N+i is defined as (x_{N+i}, y_{N+i}), the motion vector V is expressed as V (x_{N+i}−x_{N}, y_{N+1}−y_{N}). For example, if the motion estimation region on the image of the frame N+i best matched with the motion estimation region 21 on the image of the frame N is assumed as the motion estimation region 27, the motion vector of the motion estimation region 21 becomes identical to the vector toward the motion estimation region 27 from the central point of the motion estimation region 21. When the motion estimation regions of the frame N+i having mutual correlation with the motion estimation regions 22 to 26 of the frame N is assumed as 28 to 32, the motion vector can also be obtained for the motion estimation regions 22 to 26 with the same method.

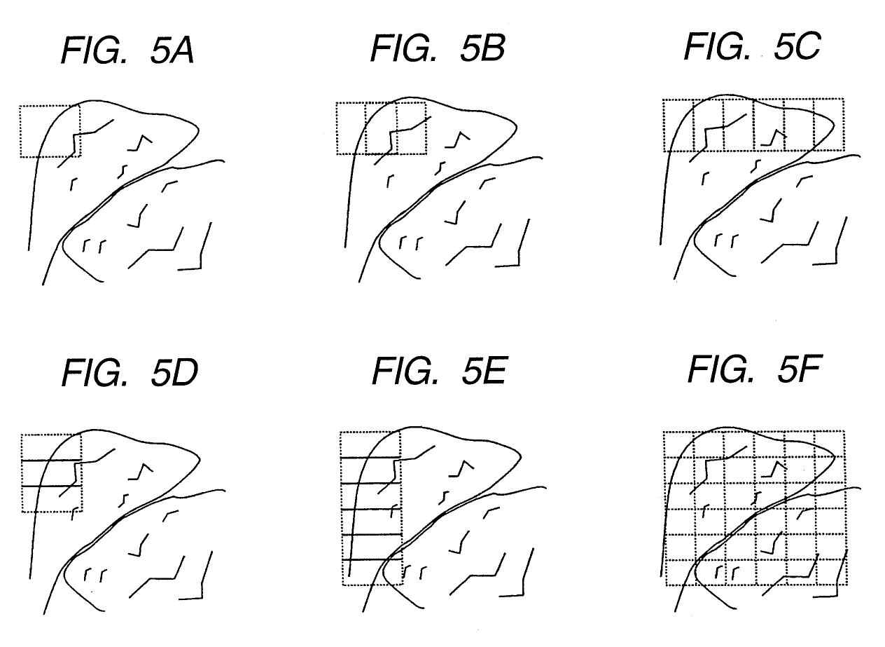

Since motion vectors should preferably be detected in detail within an image, it is actually preferable to set many motion estimation regions in the overlapping manner as shown in FIGS. 5A to 5F, although the motion estimation regions are roughly illustrated in the schematic diagrams of FIG. 3A and FIG. 3B. In FIGS. 5A to 5F, the motion estimation regions are indicated as rectangular regions surrounded with a broken line. FIG. 5A shows an example where only one motion estimation region is set. FIG. 5B shows an example where another measuring region is set additionally to result in overlapping in the horizontal direction to such motion estimation region. FIG. 5C shows an example where plural measuring regions are set in the horizontal direction in the image. FIG. 5D and FIG. 5E show examples where plural such measuring regions are set in the vertical direction. Moreover, FIG. 5F shows an example where such measuring regions are arranged in the entire part of the image. When the motion estimation region which is ith region to the right side from the left upper side and is the jth region to the lower side from the left upper side is expressed as (i, j), the motion vector corresponding to this motion estimation region can be expressed as V_{ijN}=(Vx_{ijN}, Vy_{ijN}).

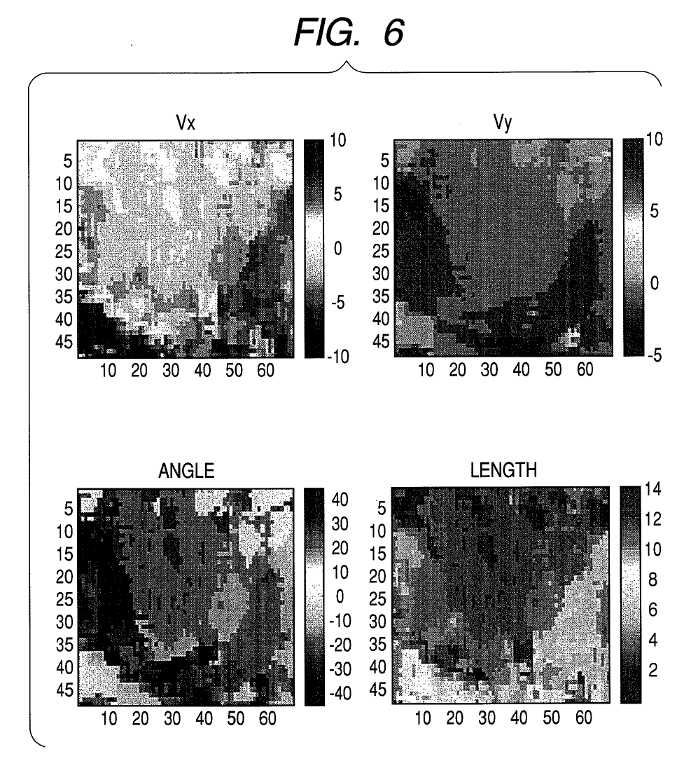

Next, a part of the motion vector where uniformity is disturbed is detected and it is determined that an edge of the object exists in this location (FIG. 2, S13). As a method for detecting disturbance in uniformity, a manipulation for converting the vector into a scalar will be required because it is difficult to make such determination for the vector quantity. In this embodiment, a scalar quantity is extracted, as shown in FIG. 6, with definition that horizontal element of motion vector is Vx, vertical element thereof is Vy, angle is θ=(Arctan(Vy/Vx)), and length L is L=(√(Vx²+Vx²)) and thereafter such scalar quantity is visualized as an image. Units are respectively pixel for Vx, Vy, and L, while degree for θ. An image of the scalar quantity extracted from the motion vector shown in FIG. 6 is computed and an edge line is obtained with the threshold value processes (S14). The threshold value is used to determine whether a scalar value of motion vector is larger or smaller than the threshold value which is defined as the value obtained by multiplying the predetermined ratio to the maximum scalar value of the image as a whole.

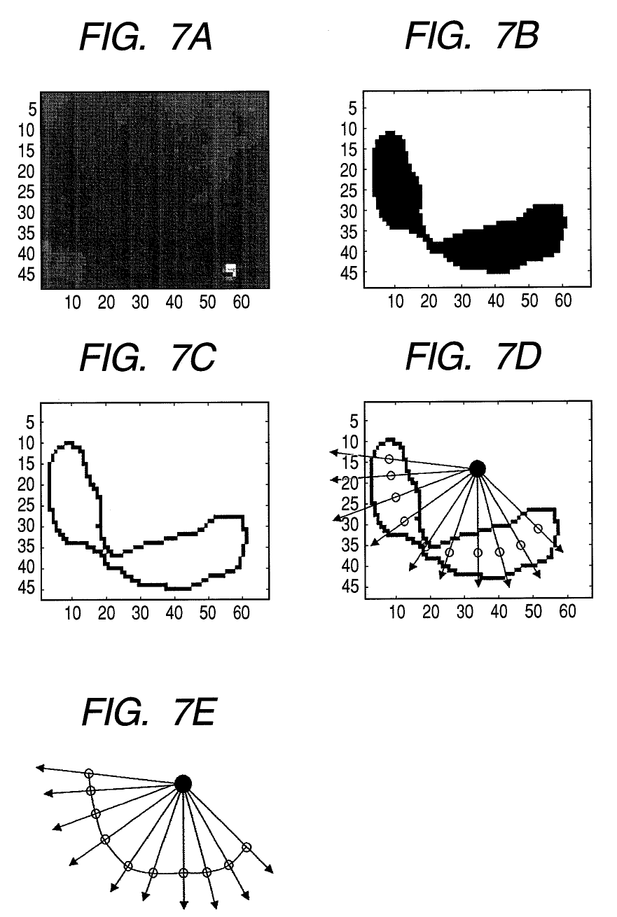

An example of process for obtaining an edge line from Vy using FIGS. 7A to 7E will be explained. First, a spatial low-pass filter is applied to the Vy data of FIG. 7A to conduct the binary process. Results are shown in FIG. 7B. Since width of edge is wide in this case, differentiation is conducted in vertical and horizontal directions, a sum of absolute values are converted to the binary values, and an edge of the edges (boundaries) having a certain width is extracted. Next, the center of edge is computed as the final edge line. As a method for such computation, a point which is assumed to exist within the region surrounded with the edges is set as shown in FIG. 7D, and lines are extended in radial in the equal interval of angle to the peripheral area from such preset point to obtain a couple of crossing points with the edges. The desired edge lines of internal organs can be obtained by obtaining the intermediate points of a couple of such crossing points as shown FIG. 7E.

If any means is not provided in this process, the edge line is not continued as the edge line or noise appears as an isolated point. Therefore, it is useful to use a filter in order to improve visibility of edge lines. As the filter, a region growing method used for detection of edge of luminance image, a method such as morphological filter, and an edge storing noise removing filter such as smoothing filter depending on direction are useful.

Moreover, there is also provided a method for improving robust property combining various scalar quantities in addition to a method for selecting only one value from those explained above as the scalar quantity. For example, an evaluation function F (Vx, Vy, θ, L)=w₁Vx+w₂Vy+w₃θ+w₄L is introduced. Here, w₁ to w₄ are weighting coefficients. Such evaluation function may be expressed by a high-order equation in place of the linear equation. Moreover, the method for obtaining the points where gradient changes to attain the edge line by obtaining gradient from distribution of the scalar quantities is also useful as the edge determining method, in addition to the method for simply determining the threshold value with the scalar quantities. For this purpose, various methods are available. For example, in one method, the vertical and horizontal elements, moreover angle and absolute value of partial differential vector are obtained for the partial differential function vector in the x and y directions of V and these values are converted into the scalar values. As explained above, the edge lines obtained by computation are displayed superimposing on the B mode cross-sectional image, elasticity image and ultrasonic blood flow image which have been obtained with the prior art method (FIG. 2, S15).

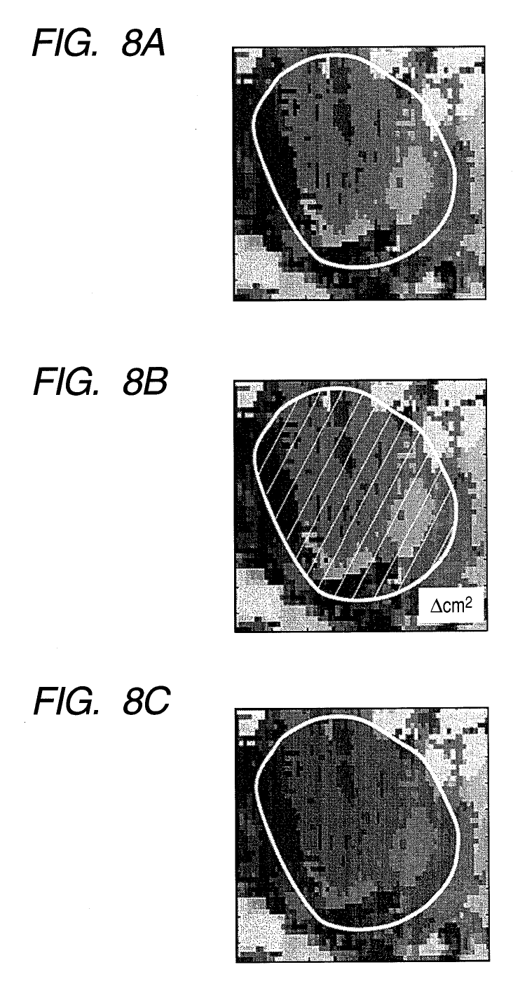

Moreover, in addition to display of the edge as image as shown in FIG. 8A, change in size of tumor can be evaluated by computing an area of the region surrounded by the edge and by outputting and displaying the results of computation as shown in FIG. 8B. Computation of area can be done with the method of prior art such as computation thereof from the number of pixels included in the region surrounded with the edge. As shown in FIG. 8C, display can also be realized by changing the color of region within the edge. Importance of evaluation in size of tumor lies in the following reasons that if the same anti-carcinoma medication is used continuously in the diagnosis using the anti-carcinoma medication, effect is gradually lowered in general and therefore such anti-carcinoma medication must be changed to the other medication, but change in size of tumor is an important measure as an index for determining whether the anti-carcinoma medication is still effective or not.

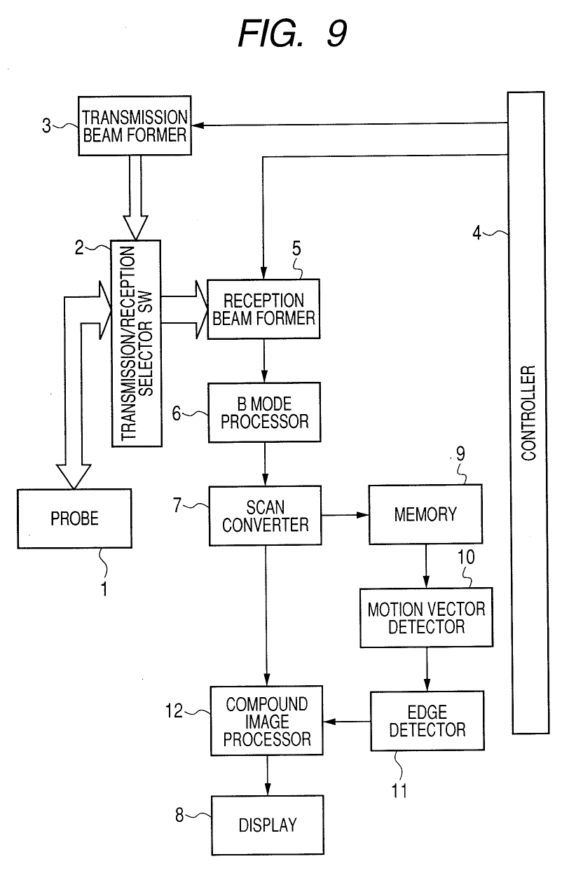

In an example of apparatus of FIG. 1, data before scan conversion is used for estimation of motion vector, but it is also possible to estimate motion vector using data after scan conversion as illustrated in an example of the apparatus structure of FIG. 9. In this case, the data after scan conversion is once stored to the memory 9 and the motion vector detector 10 conducts correlational arithmetic operation of the motion estimation regions between the frames using the data stored in the memory 9 in view of computing the motion vector. The edge detector 11 detects, on the basis of the motion vector computed by the motion vector detector 10, the edge among internal organs and the edge between the tumor and normal internal organ determined from motion within the notable image. The edge information detected by the edge detector 11 is synthesized with the image from the scan converter 7 in the compound image processor 12 and is then displayed on the display 8 as the ultrasonic cross-sectional image on which the edge image is overlapped.

Here, since it is important in the ultrasonic apparatus to display images as a real-time images in the frame rate of about 30, although not explained above in detail, increase in the estimated positions of motion vector through interpolation processes after estimation of motion vector by roughly scattering the motion estimation regions to a certain degree is also effective for high-speed computation. Motion regarding to the body motion has mainly been explained above, but the present invention can also be applied to this motion.

### Second Embodiment

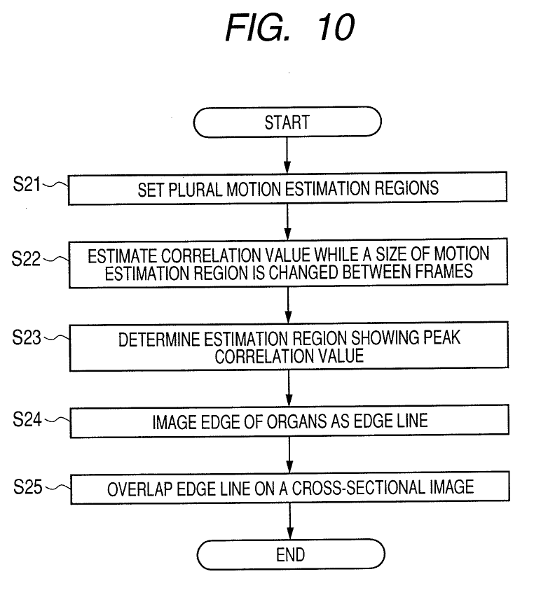

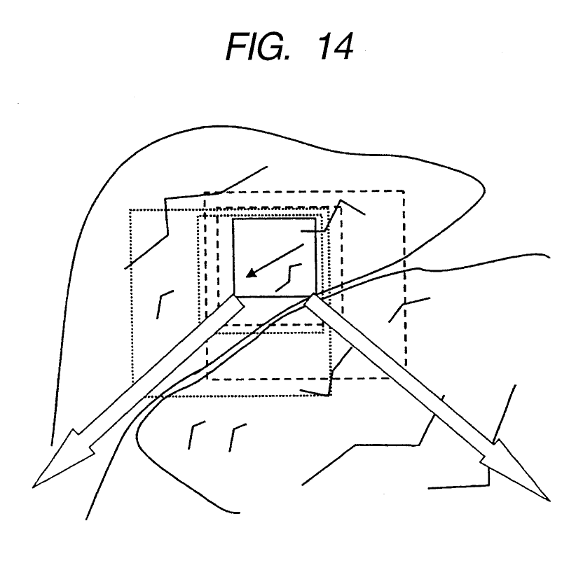

The second embodiment will be explained below from FIG. 10 with reference to FIG. 14. The ultrasonic apparatus of this embodiment may also be applied to an example of structure schematically shown in FIG. 1 or FIG. 9. However, the motion vector detector 10 conducts the operations up to measurement of correlation of the motion estimation regions between the frames and is not required to compute motion vector. Moreover, the edge detector 11 detects edges not depending on the motion vector but on the basis of shape information of the motion estimation regions when the correlation value between the frames of the motion estimation regions changes to decrease from increase.

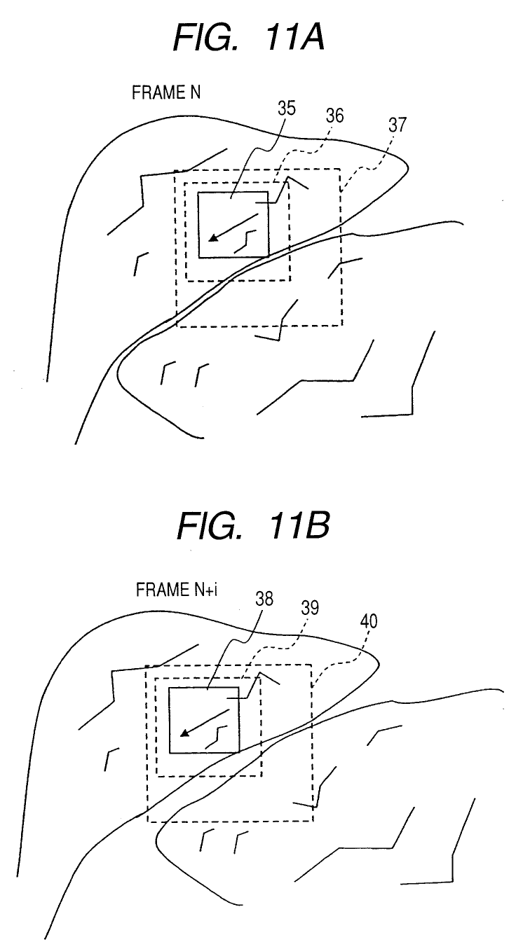

FIG. 10 is a diagram showing a flow of processes in this embodiment. First, a frame image is divided into plural motion estimation regions in view of obtaining motion vector (S21). This process is identical to the process in the step 11 in the first embodiment. Size of motion estimation region in such initial state is determined to provide a large correlation to the corresponding regions between the frames. In the second embodiment, non-continuity point of motion vector is not detected but relationship of changes in the correlation value among a couple of motion estimation regions having correlation between the size of motion estimation region and frame is used. Therefore, in the step 22, while size of the motion estimation region is increased as shown in FIGS. 11(a) and 11(b), the correlation value among the motion estimation regions having correlation between the frames is measured. FIG. 11A is a schematic diagram showing a profile to gradually increase the rectangular motion estimation region 35 set on the ultrasonic cross-sectional image of the frame N as shown by the broken lines 36 and 37. Similarly, FIG. 11B is a schematic diagram showing a profile to gradually increase the motion estimation region 38 on the ultrasonic cross-sectional image of the frame N+i having the correlation with the motion estimation region 35 on the ultrasonic cross-sectional image of the frame N as shown by the broken lines 39 and 40. When the motion estimation region increases, motion in the motion estimation region cannot be considered as uniform in a certain value of such motion estimation region and correlation among the motion estimation regions can no longer be acquired between the frames.

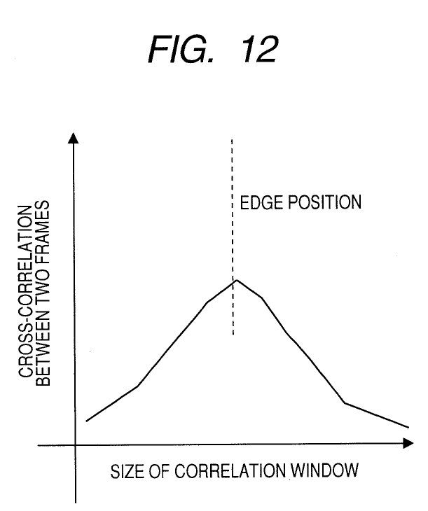

FIG. 12 shows the profile explained above using a graph. When the motion estimation region is rather small, the correlation value increases as the motion estimation region becomes larger. However, since correlation is started to be lost from an area where the motion estimation region is exceeding the edge area of motion, the correlation value starts to become small. The edge point can be determined by obtaining such changing point (peak position of the correlation value).

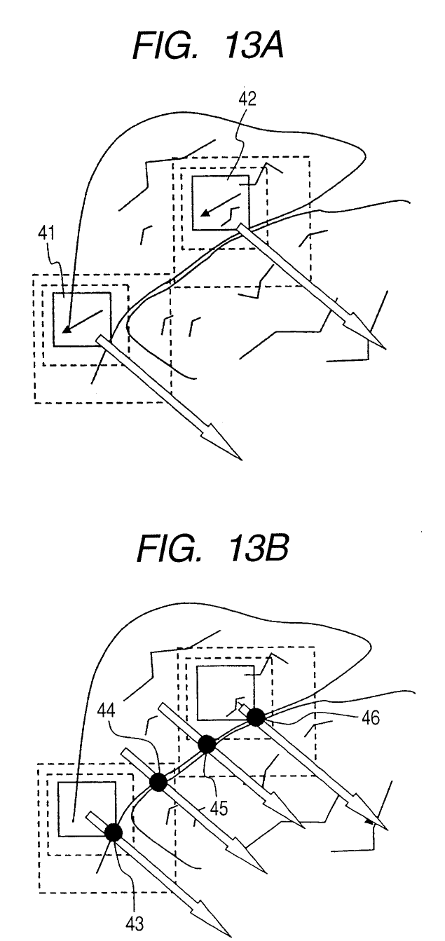

For example, as shown in FIG. 13A, while the rectangular motion estimation regions 41, 42 are widened in the right lower direction as shown by the white arrow marks, the correlation value of the motion estimation region is measured between the frames. In this case, since the correlation value of the motion estimation region between the frames changes as shown in FIG. 12 as the motion estimation region size increases, the motion estimation region when the correlation value shows the peak value is determined (FIG. 10, S23). The cross-point of the direction to wide the motion estimation region (direction indicated by the white arrow marks) and the motion estimation region when the correlation value shows the peak value, namely the right lower position in the rectangular shape in this embodiment is obtained as the point of inflexion as shown in FIG. 13B. The edge line of motion can be obtained (S24) by connecting plural points of inflexion 43 to 46 obtained for plural motion estimation regions (S24). Thereafter, the edge lines obtained are displayed superimposing on the cross-sectional image of internal organs, and the area within the edge is computed and displayed for application through change of display colors exceeding the edge of display as in the case of the first embodiment (S25).

The motion estimation region may be widened completely in the same direction as shown in FIGS. 13(a) and 13(b) or may be widened in plural directions in the setting positions of respective motion estimation regions as shown with the white arrow marks in FIG. 14. In the examples shown in the figures, after the point of inflexion is obtained by expanding first the rectangular motion estimation region in the right lower direction, another point of inflexion is obtained by sequentially widening the region in the left lower direction. Reliability is further improved in the latter case but a load of computation becomes large. In the case where plural directions for widening the motion estimation region are set, plural points of inflexion can be obtained in some cases corresponding to the direction in which the motion estimation region is widened for only one of such regions. As the shape of the motion estimation region, it may be deformed keeping its similarity as shown in the figure or the region may also be widened while the aspect ratio of the vertical and horizontal sides is changed. Here, an example of the rectangular motion estimation region has been explained but the other shape such as a circular and a polygonal shape may also be introduced as the shape of the motion estimation region.

### Third Embodiment

In the first and second embodiments, display of the edge line has been the object. However, the information obtained as a result of determination of the edges is not limited only to such object. The fact that sliding of edge is different depending on the property and shape of tumor has been known clinically. In the most obvious example, in the case of a metastatic carcinoma, since the carcinoma cell is coming from the external side, edges are easily generated against the cells initially existing in the carcinoma generating area. On the other hand, in the case of primary carcinoma such as the hepatoma, since the cells originally existing in such area change to the carcinoma, edge does not exist for the peripheral normal tissues. Moreover, even in the case of metastatic carcinoma, sliding ability of edge changes when invasion is severe or not for the peripheral tissues. In addition, when an operation has been implemented, sliding ability of edge is different because conglutination is generated.

In this embodiment, sharpness of change in motion vector distribution is effectively used as the evaluation parameter of sliding ability as a result of detection of motion vector explained in the first embodiment. Sharpness can be evaluated as the width of edge or can be evaluated as gradient in the periphery of maximal value of graph of FIG. 12 according to the method of the second embodiment. In any case, index for indicating property of carcinoma can be presented by introducing a new evaluation parameter called the sliding ability.

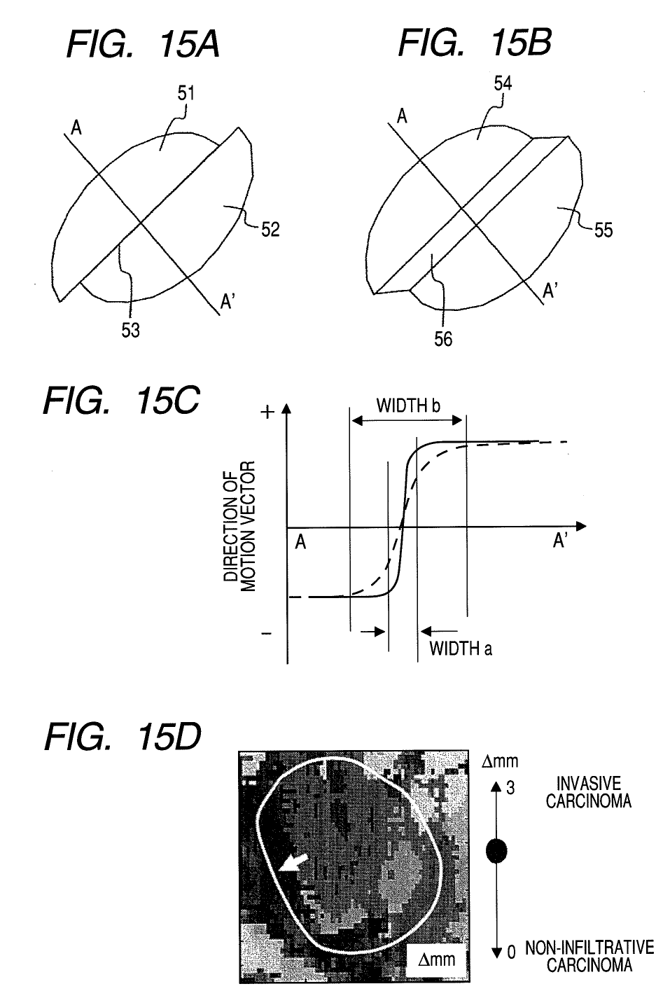

FIGS. 15A to 15D are schematic diagrams for explaining the principle of this third embodiment. FIG. 15A is a schematic diagram showing an example in the case where the edge has higher sliding ability, wherein moving direction of the adjacent tissues 51 and 52 changes sharply at the interface 53. FIG. 15B is a schematic diagram showing an example of lower sliding ability of the edge wherein a region 56 showing gradual change in the moving direction is provided between the tissues 54 and 55. Namely, direction of motion vector changes within a certain width.

FIG. 15C is a diagram where position in the direction vertical to the edge is plotted on the horizontal axis, while the direction of motion vector (element in the direction parallel to the edge of motion vector) on the vertical axis. A solid line corresponds to FIG. 15A and a broken line corresponds to FIG. 15B. When the edge has higher sliding ability as shown in FIG. 15C, change in the direction of motion vector, namely change in the element parallel to the edge of motion vector becomes sharp at the interface. Meanwhile, when the edge has lower sliding ability, change in the direction of motion vector becomes gradual. Evaluation of changing width in the direction of motion vector indicated as the widths a and b in the figure as the width of edge and collation with the result of preceding search for the carcinoma of various properties can assist estimation for property of tumor.

As the function of apparatus, it is enough when the apparatus is given the function, as shown in FIG. 15D, that changing width in the direction of motion vector is computed, conforming to the principle shown in FIG. 15C, from the motion vector on the line passing the desired position of the edge line and being vertical to the edge line when an operator designates such desired position of the edge line displayed overlapping on the ultrasonic cross-sectional image on the display 8 with a mouse or the like and the result of this computation is displayed on the display 8. In this case, it is also permissible that the line vertical to the edge line is given width in the direction along the edge line and direction of motion vector is averaged within such width. Moreover, it is also possible not only to display the width of edge but also to display an example of the typical tumor of each corresponding organs on the scale as shown in the right side of FIG. 15D in view of assisting estimation of property of the carcinoma displayed as the image. Width of the measured edge can be displayed as a black point on the scale.

### Fourth Embodiment

In this embodiment, edge can be detected stably by utilizing the information about plural frames.

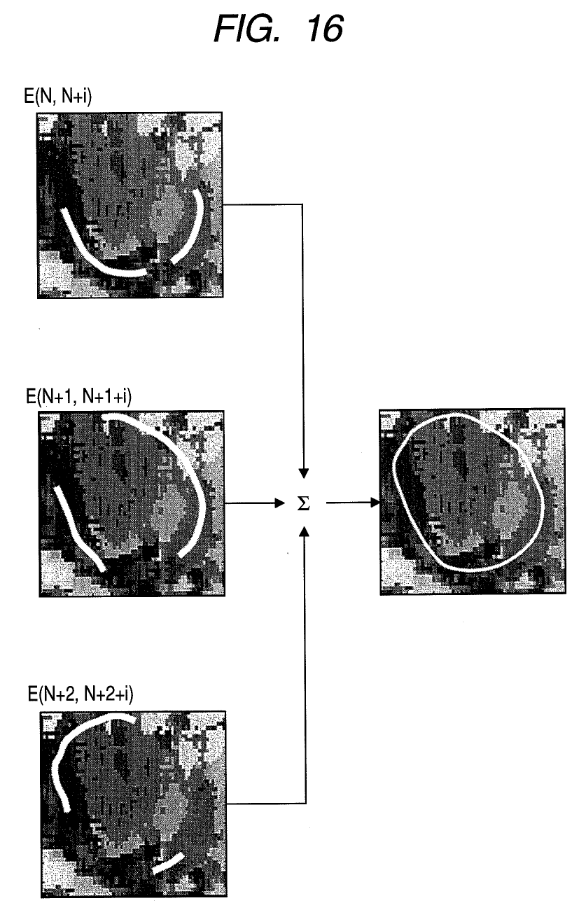

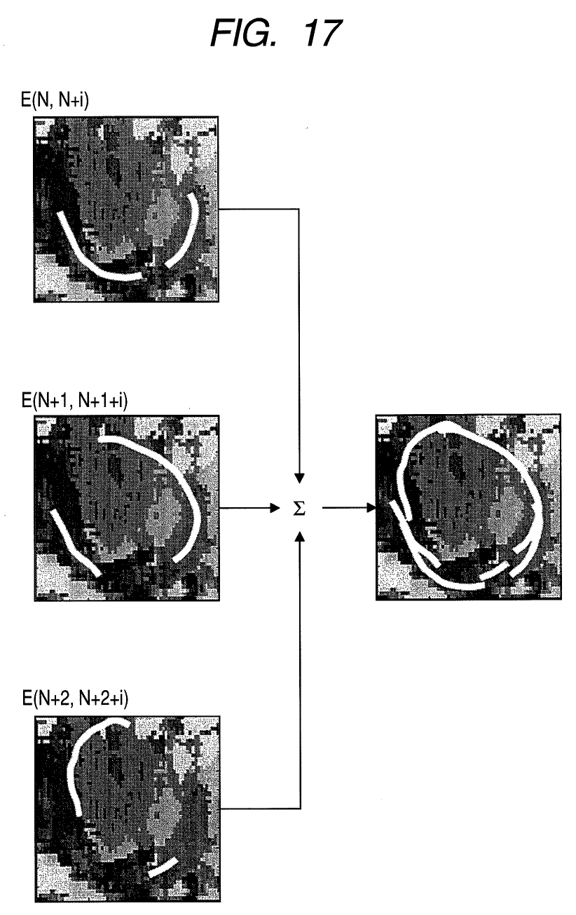

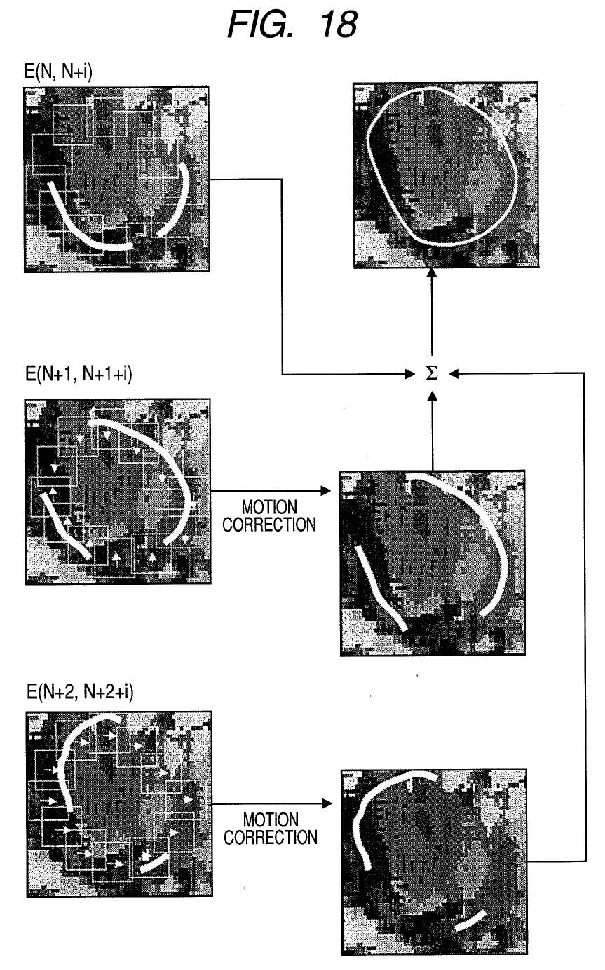

Concept will be explained first as follows. The edge obtained using the frames N and N+1 is expressed as E(N, N+1). Stability of edge extraction can be improved by simply conduction addition of edges E(N, N+1)+E(N+1, N+2)+E(N+2, N+3)+ . . . , but the edge is blurred due to accumulation. The state where the edge is never blurred due to the addition will be explained with reference to FIG. 16. When motion is caused by breathing or external pressure, all edges are not sliding. The best extracted edge is different respectively in the edges E(N, N+1), E(N+1, N+2), and E(N+2, N+3). The edges can be seen continuous by adding these edges. However, as is already explained above, when the edges are only added simply, the edge may become discontinuous or may be blurred as shown in FIG. 17. On the contrary, there is proposed a method for obtaining motion vector between frames to compensate and add these vectors. For example, as shown in FIG. 18, the motion estimation regions are obtained and motion vectors among these regions are also obtained. Motion of edge E(N+1, N+2) is corrected and then motion of edge E(N+2, N+3) is also corrected. Stable edge extraction can be realized, while effect of blur is controlled, by repeating overlapping of the motion estimation region on the basis of the result of such correction.

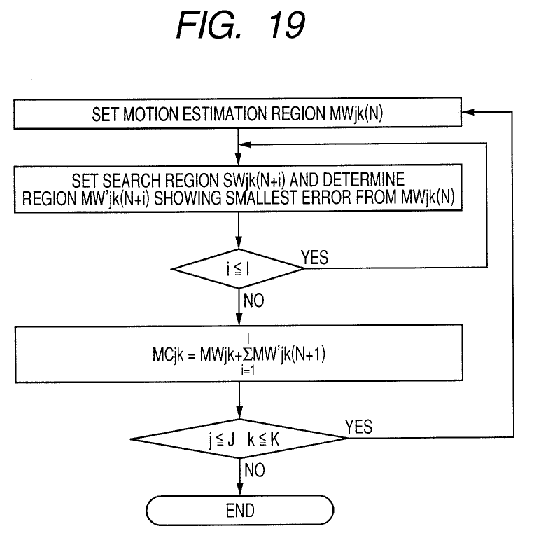

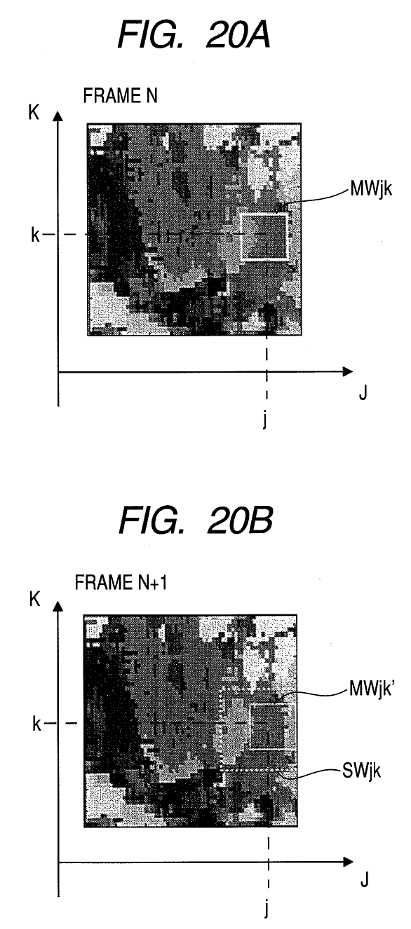

A method for accumulation of correction for motion between frames will be explained in more detail with reference to the flowchart of FIG. 19 and FIGS. 20(a) and 20(b). In the case where the images of frame N and frame N+1 are accumulated through correction in motion as shown in FIGS. 20(a) and 20(b), a motion estimation region MWⱼₖ(N) around the coordinate (j, k) is set first within the frame N. Next, a wide search region SWⱼₖ(N+1) which is wider in the right and left upper and lower directions from the motion estimation region MWⱼₖ(N) is set in the frame N+1. The center coordinate (j,k) of the search region is identical to the center coordinate of MWⱼₖ(N) and the size of the same search region is set larger than MWjk(N) in such a degree to consider that the estimation object moves between the frames. Next, the region MW′jk(N+1) in the same size as MWjk(N) is set in this search region SWjk(N+1) and then following computation is conducted.

Σ(MWⱼₖ(N)−MW′ⱼₖ(N+1))²

MW′ⱼₖ(N+1) for minimizing Σ(MWⱼₖ(N)−MW′ⱼₖ(N+1))² is obtained by fully moving MW′ⱼₖ(N+1) within SWⱼₖ(N+1). Here, MW′ⱼₖ(N+1) is added to MWⱼₖ(N). When the number of frames to be added is 1, above operations are conducted until the frame N+1 and moreover the region is moved to the entire part of image regarding j and k. This operation realizes addition of the motion correction frames. When equal result is obtained, sequence in the flowchart is not always required to be identical to that in FIG. 19. In addition, an example of square sum of difference has been explained above, but the absolute value of difference can also be considered and the other arithmetic operation such as tow-dimensional convolution can also be conducted.

One motion estimation region MWjk(N) can be set on the image of edge E(N, N+1) estimated using the frames N and N+1 by combining such motion compensating accumulation and edge extraction. Next, the search region SWⱼₖ(N+I, N+i+1) which is wider in the right and left directions from the position corresponding to MWⱼₖ(N, N+1) is set on the image of edge E(N+I, N+i+1). A value of MWⱼₖ(N+i, N+i+1) for minimizing the square sum of difference is obtained by repeating the steps for setting the region MW′ⱼₖ(N+I, N+i+1) and for computing the square sum of difference from MWⱼₖ(N, N+1), until the region MW′ⱼₖ(N+i, N+i+1) scans the total area of SWⱼₖ(N+i, N+i+1). The value obtained is then added to MWⱼₖ(N, N+1). This scanning is conducted while i is changed until the predetermined number of frames to be added becomes equal to 1. Moreover, the motion compensating accumulation between frames can be realized by scanning the entire part of image in regard to j and k. Since the edge E(N, N+1) includes the information of both N and N+1 of the original image, MWjk(N, N+1) may use the average value of the frames N and N+1 or only the data of one of these frames. When edge extraction is conducted for N and N+i (i>1), any of the average value, weighted sum, or representative value of all data between the frames N and N+i may be used. Such motion compensating accumulation can realize stable edge traction as shown in FIG. 18.

What is claimed is:

1. An ultrasonic apparatus, comprising:
an ultrasonic cross-sectional image acquirer that acquires, on the time series basis, a plurality of frames of the ultrasonic cross-sectional images of an inspection object;
a memory that stores said ultrasonic cross-sectional images of a plurality of frames acquired;
a motion detector that extracts information about motions of each tissue within the ultrasonic cross-sectional images of a first frame by comparing the ultrasonic cross-sectional images of said first frame with ultrasonic cross-sectional images of a second frame read from said memory;
an edge detector that detects an edge within said ultrasonic cross-sectional images on the basis of the information about motion detected with said motion detector; and
a display that displays the edge detected with said edge detector overlapping on the ultrasonic cross-sectional images acquired with said ultrasonic cross-sectional image acquirer.

2. The ultrasonic apparatus according to claim 1, wherein information about area surrounded with said edge is displayed on said display.

3. The ultrasonic apparatus according to claim 1, wherein the ultrasonic cross-sectional images in the internal and external sides of said edge are discriminated and displayed on said display.

4. The ultrasonic apparatus according to claim 1, wherein said motion detector respectively sets a plurality of estimation regions on the ultrasonic cross-sectional images of the first frame and the second frame read from said memory, detects estimation regions of the second frame matched with estimation regions of the first frame through pattern matching, and extracts direction and size of motion of each tissue from the relative positions of the estimation region of said first frame and the estimation region of said second frame matched therewith.

5. The ultrasonic apparatus according to claim 4, wherein said edge detector obtains an edge by conducting threshold value process to images formed on scalar quantity extracted from the information about motion of each tissue within said ultrasonic cross-sectional images.

6. The ultrasonic apparatus according to claim 1, wherein said motion detector obtains the estimation region when a correlation value shows the peak value by respectively setting a plurality of estimation regions on the ultrasonic cross-sectional images of the first frame and the ultrasonic cross-sectional images of the second frame read from said memory and by detecting said correlation value of the estimation region of said first frame and the estimation region of said second frame matched therewith through pattern matching while sizes of the estimation region of said first frame and the estimation region of said second frame are expanded in the predetermined direction.

7. The ultrasonic apparatus according to claim 6, wherein said edge detector defines a cross point between the estimation region and said predetermined direction when said correlation values shows the peak as the point of inflexion and detects said edge by connecting a plurality of points of inflexion.

8. The ultrasonic apparatus according to claim 6, wherein said estimation region is formed in the rectangular shape and size of said estimation region is expanded in the manner that one crest point of such rectangular shape moves long said preset direction.

9. The ultrasonic apparatus according to claim 6, wherein a plurality of directions are set for expanding size of said estimation region.

10. The ultrasonic apparatus according to claim 1, wherein said ultrasonic cross-sectional image acquirer acquires said frames for a plurality of regions, said edge detector detects and compensates the edge of each region of a plurality of estimation regions, and said display displays the edges corrected for each of a plurality of estimation regions overlapping on the ultrasonic cross-sectional images acquired with said ultrasonic cross-sectional image acquirer.
